SEQUENCE LISTING (4 sequences)

SEQ ID NO: 1 - DNA, 818 bp, synthetic construct
  source 1..818: mol_type = other DNA
gaattcgccaccatggactggacctggatcctgttcctggtggccgccgccacacgggtgcacagcttccaggacccccaggagagcggcagaaagctgcctcagctgtgtaccgagctgcagaccaccatccacgacatcatcctggagtgtgtgtactgtaagcagcagctgctgaggagagaggtgtacgaccgggacctgtgtatcgtgtacagggacggcaatccctacgccgtgtgtgacaagtgcctgaagttctacagcaagatcagcgagtaccggcactactgctacagcctgtacggcaccaccctggagcagcagtacaacaagcccctgtgtgacctgctgatccggtgtatcaactgccagaagcccctgcagagacacctggacaagaagcagcggttccacaacatcaggggcagatggaccggcagatgtatgagctgctgccggagcagcagaaccagaagggagacccagctgagaggccggaagagaagaagccacggcgatacccccaccctgcacgagtacatgctggacctgcagcctgagaccaccgatctgtacggctacggccagctgaatgacagcagcgaggaggaggatgagatcgacggccctgccggccaggccgagcccgacagagcccactacaacatcgtgaccttttgctgtaagtgtgacagcaccctgagactgtgcgtgcagagcacccacgtggacatcagaaccctggaggatctgctgatgggcaccctgggcatcgtgtgtcccatctgctcccagaaaccctgatgagcggccgc

SEQ ID NO: 2 - DNA, 837 bp, synthetic construct
  source 1..837: mol_type = other DNA
atggactggacctggatcctgttcctggtggccgctgccacacgggtgcacagcgccagattcgaggaccccaccaggagcggctacaagctgcccgatctgtgtaccgagctgaacaccagcctgcaggacatcgagatcacctgtgtgtactgtaagaccgtgctggagctgaccgaggtgttcgagaaggacctgttcgtggtgtacagggacagcatcccccacgccgcctgccacaagtgtatcgacttctacagccggatccgggagctgagacactacagcgacagcgtgtacggcgataccctggagaagctgaccaacaccggcctgtacaacctgctgatccggtgcctgagatgccagaagcccctgctgagacacctgaacgagaagcggcggttccacaacatcgccggccactacagaggccagtgccacagctgctgtaacagggccaggcaggagagactgcagcggagaagagagacccaggtgaggggcaggaagagaagaagccacggccccaaggccaccctgcaggatatcgtgctgcacctggagccccagaatgagatccccgtggatctgctgggccacggccagctgtccgacagcgaggaggagaacgacgagatcgacggcgtgaatcaccagcacctgcctgccagaagagccgagcctcagaggcacaccatgctgtgtatgtgctgtaagtgtgaggcccggatcgaactggtggtggagagcagcgccgacgacctgagagccttccagcagctgttcctgaacaccctgagcttcgtgtgtccttggtgtgccagccagcagtga

SEQ ID NO: 3 - protein, 264 aa, synthetic construct
MDWTWILFLVAAATRVHSFQDPQESGRKLPQLCTELQTTIHDIILECVYCKQQLLRREVYDRDLCIVYRDGNPYAVCDKCLKFYSKISEYRHYCYSLYGTTLEQQYNKPLCDLLIRCINCQKPLQRHLDKKQRFHNIRGRWTGRCMSCCRSSRTRRETQLRGRKRRSHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKP

SEQ ID NO: 4 - protein, 278 aa, synthetic construct
MDWTWILFLVAAATRVHSARFEDPTRSGYKLPDLCTELNTSLQDIEITCVYCKTVLELTEVFEKDLFVVYRDSIPHAACHKCIDFYSRIRELRHYSDSVYGDTLEKLTNTGLYNLLIRCLRCQKPLLRHLNEKRRFHNIAGHYRGQCHSCCNRARQERLQRRRETQVRGRKRRSHGPKATLQDIVLHLEPQNEIPVDLLGHGQLSDSEEENDEIDGVNHQHLPARRAEPQRHTMLCMCCKCEARIELVVESSADDLRAFQQLFLNTLSFVCPWCASQQ

(19) United States
(12) Patent Application Publication
(10) Pub. No.: US 2024/0293528 A1
(43) Pub. Date: Sep. 5, 2024

(54) METHODS OF TREATMENT OF HIGH-GRADE SQUAMOUS INTRAEPITHELIAL LESION (HSIL)

(71) Applicant: Inovio Pharmaceuticals, Inc., Plymouth Meeting, PA (US)

(72) Inventors: Kimberly A. Kraynyak, Norristown, PA (US); Jean D. Boyer, Yardley, PA (US); Matthew P. Morrow, Malvern, PA (US); Prakash Bhuyan, Lafayette Hill, PA (US)

(21) Appl. No.: 18/593,320

(22) Filed: Mar. 1, 2024

Related U.S. Application Data
(60) Provisional application No. 63/487,843, filed on Mar. 1, 2023.

(51) Int. Cl. A61K 39/12; A61K 39/00; A61K 47/02; A61K 47/12; A61P 31/20; C12N 7/00; C12Q 1/70

(52) U.S. Cl. CPC A61K 39/12; A61K 47/02; A61K 47/12; A61P 31/20; C12N 7/00; C12Q 1/70; A61K 2039/545; A61K 2039/572; A61K 2039/575; C12N 2710/20034

(57) ABSTRACT

Described are methods of treating human papillomavirus (HPV) type 16- or HPV type 18-related High-grade Squamous Intraepithelial Lesion (HSIL) of the cervix

### CROSS-REFERENCE TO RELATED APPLICATIONS

This application claims the benefit of U.S. Provisional Patent Application No. 63/487,843, filed Mar. 1, 2023, the disclosure of which is incorporated herein by reference it its entirety.

### SEQUENCE LISTING

The instant application contains a Sequence Listing which is being submitted herewith electronically in XML format and is hereby incorporated by reference in its entirety. Said XML copy, created on Mar. 15, 2024, is named 104409000947_Sequence Listing.xml and is 6,789 bytes in size.

### TECHNICAL FIELD

Provided are methods of treating human papillomavirus (HPV) type 16- or HPV type 18-related High-grade Squamous Intraepithelial Lesion (HSIL) of the cervix.

### BACKGROUND

Infection with human papillomavirus (HPV) can lead to malignant neoplasia, localized primarily in the ano-genital area and aero-digestive tract, in both men and women. HPV types tropic to mucosal tissues are classified into high-risk (HR), based on their potential to cause cancer, and low-risk (LR) (causing generally benign lesions). HPV-16 and HPV-18 are the most significant amongst high-risk types since they are responsible for most HPV-caused cancers. In the US alone, approximately 14 million new genital HPV infections occur annually, and approximately half of these infections are with a high-risk HPV type. Up to 13,000 women in the US alone are diagnosed with cervical cancer each year, with an estimated 4,120 deaths annually in year 2017. HPV-16 is the most common high-risk genotype and combined with HPV-18 these two genotypes are estimated to cause about 70% of all cervical cancers.

Incident infection by HPV is characterized by ongoing viral replication and shedding and is associated with early histologic changes (grade 1 cervical intraepithelial neoplasia) when the female cervix is infected with HPV. Most cases of genital HPV infection clear spontaneously, but persistent infection with one or more oncogenic (high-risk) HPV genotypes can lead to the development of precancerous, histologic high grade squamous intraepithelial lesions of the cervix, HSIL which is inclusive of grade 2 and 3 cervical intraepithelial neoplasia (CIN2/3). Over time, typically years, cervical HSIL can progress to invasive cancer of the cervix. The basis for these changes is attributed to the viral proteins E6 and E7. Infected cells produce E6 and E7 constitutively which increases the degradation of cell cycle regulation proteins p53 and pRb, respectively, resulting in unrestricted cell growth and neoplasia.

There is a need for treatments for HPV16- or HPV18-related HSIL.

### SUMMARY

Provided herein are methods of treating human papillomavirus (HPV) type 16- or HPV type 18-related high grade cervical intraepithelial lesion (HSIL), said method comprising administering a therapeutically effective amount of VGX-3100 a subject in need thereof.

In additional embodiments, the HPV type 16- or HPV type 18-related HSIL of the cervix is determined by a biopsy.

In some embodiments, the VGX-3100 is administered to the subject by intramuscular injection followed by electroporation. In certain embodiments, the VGX-3100 is administered to the subject at a dose of 6 mg. In further embodiments, the VGX-3100 is administered to the subject three times over the course of 12 weeks. In still further embodiments, the VGX-3100 is formulated at a concentration of 6 mg/ml in 150 mM sodium chloride and 15 mM sodium citrate.

In certain embodiments, administration of VGX-3100 results in virologic clearance of HPV-16 and/or HPV-18 and histopathologic regression of cervical HSIL. In further embodiments, administration of VGX-3100 results in histopathologic regression of cervical HSIL. In still further embodiments, administration of VGX-3100 results in virologic clearance of HPV-16 and/or HPV-18. In certain embodiments, administration of VGX-3100 results in complete histopathologic regression of cervical HSIL to normal. In further embodiments, administration of VGX-3100 results in complete histopathologic regression of cervical HSIL to normal and virologic clearance of HPV-16 and/or HPV-18. In still further embodiments, administration of VGX-3100 results in histopathologic nonprogression. In certain embodiments, administration of VGX-3100 results in clearance of HPV-16 and/or HPV-18 infection from noncervical anatomic locations. In further embodiments, administration of VGX-3100 results in improved humoral and cellular immune response to VGX-3100 following a third administration of VGX-3100 and at 36 weeks following administration of VGX-3100 as assessed relative to baseline.

In certain embodiments, the result of VGX-3100 administration is evaluated at 36 weeks following administration of VGX-3100.

### DETAILED DESCRIPTION OF ILLUSTRATIVE EMBODIMENTS

The disclosed methods may be understood more readily by reference to the following detailed description, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

When a range of numerical values is expressed, another embodiment includes from the one particular value and/or to the other particular value. Further, reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods, which are, for clarity, described herein in the context of separate embodiments, may also be provided in combination in a single embodiment. Conversely, various features of the disclosed methods that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include the plural.

### Certain Terminology

As used herein, the term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

As used herein, the term “at least one” means “one or more.”

As used herein, the term “subject” as used herein refers to any animal, but in particular humans. Thus, the methods are applicable to human and nonhuman animals, although preferably used most preferably with humans. “Subject” and “patient” are used interchangeably herein.

As used herein, the term “comprising” is intended to include examples encompassed by the terms “consisting essentially of” and “consisting of”: similarly, the term “consisting essentially of” is intended to include examples encompassed by the term “consisting of.”

As used herein, “treating” and like terms refer to reducing the severity and/or frequency of human papillomavirus (HPV) type 16- or HPV type 18-related high grade cervical intraepithelial lesion (HSIL) of the cervix symptoms for example and cervical high grade squamous intraepithelial lesions (HSIL) lesions: eliminating HPV type 16 or HPV type 18 infection symptoms, especially HSIL lesions; and/or clearing HPV type 16 or HPV type 18 virus from the subject; and/or resolution to cervical low grade squamous intraepithelial lesions (LSIL) or normal tissue.

As used herein, the term “coding sequence” or “encoding nucleic acid” may mean refers to the nucleic acid (RNA or DNA molecule) that comprise a nucleotide sequence which encodes a polypeptide. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides, e.g., an IgE leader sequence.

As used herein, the term “nucleic acid” or “oligonucleotide” or “polynucleotide” may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

As used herein, the term “operably linked” may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function,

As used herein, the term “promoter” may mean a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

As used herein, the term “vector” “may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

As used herein, the term “adverse event” (AE) is defined according to the Common Toxicity Criteria for Adverse Events (CTCAE) Version 4.03 grading scale. The investigator graded the laboratory AEs and clinical AEs with respect to the following levels of severity as per CTCAE Version 4.03 for applicable subject populations: grade 1 (mild), grade 2 (moderate), grade 3 (severe), grade 4 (potentially life-threatening), and grade 5 (death).

As used herein, the term “responder” refers to study subjects who met the primary endpoint of proportion of baseline biomarker-positive women with no evidence of cervical HSIL on histology sample and no evidence of HPV-16 and/or HPV-18 in cervical samples by type specific HPV testing at Week 36 visit.

As used herein, the term “placebo” means administration of a pharmaceutical composition that does not include VGX-3100.

### Methods of Treatment and Diagnosis

Provided herein are methods of treating human papillomavirus (HPV) type 16- or HPV type 18-related high grade cervical intraepithelial lesion (HSIL), said method comprising, consisting of, or consisting essentially of administering a therapeutically effective amount of VGX-3100 to a subject in need thereof.

Also provided herein is VGX-3100 for use in a method of treating human papillomavirus (HPV) type 16- or HPV type 18-related high grade cervical intraepithelial lesion (HSIL), said method comprising, consisting of, or consisting essentially of administering a therapeutically effective amount of VGX-3100 to a subject in need thereof.

Also provided herein are uses of VGX-3100 in the manufacture of a medicament for treating human papillomavirus (HPV) type 16- or HPV type 18-related high grade cervical intraepithelial lesion (HSIL), said method comprising, consisting of, or consisting essentially of administering a therapeutically effective amount of VGX-3100 to a subject in need thereof.

In additional embodiments, the HPV type 16- or HPV type 18-related HSIL of the cervix is determined by a biopsy.

In certain embodiments, administration of VGX-3100 results in virologic clearance of HPV-16 and/or HPV-18 and histopathologic regression of cervical HSIL. In further embodiments, administration of VGX-3100 results in histopathologic regression of cervical HSIL. In still further embodiments, administration of VGX-3100 results in virologic clearance of HPV-16 and/or HPV-18. In certain embodiments, administration of VGX-3100 results in complete histopathologic regression of cervical HSIL to normal. In further embodiments, administration of VGX-3100 results in complete histopathologic regression of cervical HSIL to normal and virologic clearance of HPV-16 and/or HPV-18. In still further embodiments, administration of VGX-3100 results in histopathologic nonprogression. In certain embodiments, administration of VGX-3100 results in clearance of HPV-16 and/or HPV-18 infection from noncervical anatomic locations. In further embodiments, administration of VGX-3100 results in improved humoral and cellular immune response to VGX-3100 following a third administration of VGX-3100 and at 36 weeks following administration of VGX-3100 as assessed relative to baseline.

Also provided herein are methods of improving virologic clearance of HPV-16 and/or HPV-18 and histopathologic regression of cervical HSIL in a subject comprising, consisting of, or consisting essentially of administering a therapeutically effective amount of VGX-3100 to a subject in need thereof. In certain embodiments, the improvement in virologic clearance of HPV-16 and/or HPV-18 and histopathologic regression of cervical HSIL is relative to administration of a placebo to a subject or a population of subjects. In certain embodiments, the improvement in virologic clearance of HPV-16 and/or HPV-18 and histopathologic regression of cervical HSIL is relative to no treatment of a subject or a population of subjects. In certain embodiments, the improvement in virologic clearance of HPV-16 and/or HPV-18 and histopathologic regression of cervical HSIL is relative to treatment of a subject of population of subjects with the standard of care.

Also provided herein are methods of improving histopathologic regression of cervical HSIL in a subject comprising, consisting of, or consisting essentially of administering a therapeutically effective amount of VGX-3100 to a subject in need thereof. In certain embodiments, the improvement in histopathologic regression of cervical HSIL is relative to administration of a placebo to a subject or a population of subjects. In certain embodiments, the improvement in histopathologic regression of cervical HSIL is relative to no treatment of a subject or a population of subjects. In certain embodiments, the improvement in histopathologic regression of cervical HSIL is relative to treatment of a subject of population of subjects with the standard of care.

Also provided herein are methods of achieving complete histopathologic regression of cervical HSIL to normal in a subject comprising, consisting of, or consisting essentially of administering a therapeutically effective amount of VGX-3100 to a subject in need thereof. In certain embodiments, the achievement of complete histopathologic regression of cervical HSIL to normal is relative to administration of a placebo to a subject or a population of subjects. In certain embodiments, the achievement of complete histopathologic regression of cervical HSIL is relative to no treatment of a subject or a population of subjects. In certain embodiments, the achievement of complete histopathologic regression of cervical HSIL is relative to treatment of a subject of population of subjects with the standard of care.

Also provided herein are methods of achieving complete histopathologic regression of cervical HSIL to normal and virologic clearance of HPV-16 and/or HPV-18 in a subject comprising, consisting of, or consisting essentially of administering a therapeutically effective amount of VGX-3100 to a subject in need thereof. In certain embodiments, the achievement of complete histopathologic regression of cervical HSIL to normal and virologic clearance of HPV-16 and/or HPV-18 is relative to administration of a placebo to a subject or a population of subjects. In certain embodiments, the achievement of complete histopathologic regression of cervical HSIL to normal and virologic clearance of HPV-16 and/or HPV-18 is relative to no treatment of a subject or a population of subjects. In certain embodiments, the achievement of complete histopathologic regression of cervical HSIL to normal and virologic clearance of HPV-16 and/or HPV-18 is relative to treatment of a subject of population of subjects with the standard of care.

Also provided herein are methods of improving histopathologic non-progression in a subject comprising, consisting of, or consisting essentially of administering a therapeutically effective amount of VGX-3100 to a subject in need thereof. In certain embodiments, the improvement in histopathologic non-progression is relative to administration of a placebo to a subject or a population of subjects. In certain embodiments, the improvement in histopathologic non-progression is relative to no treatment of a subject or a population of subjects. In certain embodiments, the improvement in histopathologic non-progression is relative to treatment of a subject of population of subjects with the standard of care.

Also provided herein are methods of improving clearance of HPV-16 and/or HPV-18 infection from noncervical anatomic locations in a subject comprising, consisting of, or consisting essentially of administering a therapeutically effective amount of VGX-3100 to a subject in need thereof. In certain embodiments, the improvement in clearance of HPV-16 and/or HPV-18 infection from noncervical anatomic locations is relative to administration of a placebo to a subject or a population of subjects. In certain embodiments, the improvement in clearance of HPV-16 and/or HPV-18 infection from noncervical anatomic locations is relative to no treatment of a subject or a population of subjects. In certain embodiments, the improvement in clearance of HPV-16 and/or HPV-18 infection from noncervical anatomic locations is relative to treatment of a subject of population of subjects with the standard of care.

Also provided herein are methods of improving humoral and cellular immune response to VGX-3100 following a third administration of VGX-3100 and at 36 weeks following administration of VGX-3100 as assessed relative to baseline in a subject comprising, consisting of, or consisting essentially of administering a therapeutically effective amount of VGX-3100 a subject in need thereof. In certain embodiments, the improvement in clearance of HPV-16 and/or HPV-18 infection from noncervical anatomic locations is relative to administration of a placebo to a subject or a population of subjects. In certain embodiments, the improvement in humoral and cellular immune response to VGX-3100 following a third administration of VGX-3100 and at 36 weeks following administration of VGX-3100 as assessed relative to baseline is relative to no treatment of a subject or a population of subjects. In certain embodiments, the improvement in humoral and cellular immune response to VGX-3100 following a third administration of VGX-3100 and at 36 weeks following administration of VGX-3100 as assessed relative to baseline is relative to treatment of a subject of population of subjects with the standard of care.

In certain embodiments, the result of VGX-3100 administration or the improvement achieved by VGX-3100 administration is evaluated at 36 weeks following administration of VGX-3100.

### Routes of Administration and Pharmaceutical Compositions

VGX-3100 may be delivered using any of several well-known technologies including DNA injection (also referred to as DNA vaccination), recombinant vectors such as recombinant adenovirus, recombinant adenovirus related virus and recombinant vaccinia.

Routes of administration include, but are not limited to, intramuscular, intranasally, intraperitoneal, intradermal, subcutaneous, intravenous, intraarterially, intraocularly and oral as well as topically, transdermally, by inhalation or suppository or to mucosal tissue such as by lavage to vaginal, rectal, urethral, buccal and sublingual tissue. Preferred routes of administration include intramuscular, intraperitoneal, intradermal and subcutaneous injection. Genetic constructs may be administered by means including, but not limited to, electroporation methods and devices, traditional syringes, needleless injection devices, or “microprojectile bombardment gone guns”. In some embodiments, the VGX-3100 is administered to the subject by intramuscular injection. In certain embodiments, the VGX-3100 is administered to the subject by intramuscular injection followed by electroporation.

Examples of electroporation devices and electroporation methods preferred for facilitating delivery of the DNA vaccines, include those described in U.S. Pat. No. 7,245,963 by Draghia-Akli, et al, U.S. Patent Pub. 2005/0052630 submitted by Smith, et al., the contents of which are hereby incorporated by reference in their entirety. Also preferred, are electroporation devices and electroporation methods for facilitating delivery of the DNA vaccines provided in co-pending and co-owned U.S. patent application Ser. No. 11/874,072, filed Oct. 17, 2007, which claims the benefit under 35 USC 1 19(e) to U.S. Provisional Applications Ser. No. 60/852,149, filed Oct. 17, 2006, and 60/978,982, filed Oct. 10, 2007, all of which are hereby incorporated in their entirety. In certain embodiments, the electroporation device is a CELLECTRA®-5P device.

The following is an example of an embodiment using electroporation technology and is discussed in more detail in the patent references discussed above: electroporation devices can be configured to deliver to a desired tissue of a mammal a pulse of energy producing a constant current similar to a preset current input by a user. The electroporation device comprises an electroporation component and an electrode assembly or handle assembly. The electroporation component can include and incorporate one or more of the various elements of the electroporation devices, including: controller, current waveform generator, impedance tester, waveform logger, input element, status reporting element, communication port, memory component, power source, and power switch. The electroporation component can function as one element of the electroporation devices, and the other elements are separate elements (or components) in communication with the electroporation component. In some embodiments, the electroporation component can function as more than one element of the electroporation devices, which can be in communication with still other elements of the electroporation devices separate from the electroporation component. The use of electroporation technology to deliver the improved HPV vaccine is not limited by the elements of the electroporation devices existing as parts of one electromechanical or mechanical device, as the elements can function as one device or as separate elements in communication with one another. The electroporation component is capable of delivering the pulse of energy that produces the constant current in the desired tissue and includes a feedback mechanism. The electrode assembly includes an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism can receive the measured impedance and can adjust the pulse of energy delivered by the electroporation component to maintain the constant current.

In some embodiments, the plurality of electrodes can deliver the pulse of energy in a decentralized pattern. In some embodiments, the plurality of electrodes can deliver the pulse of energy in the decentralized pattern through the control of the electrodes under a programmed sequence, and the programmed sequence is input by a user to the electroporation component. In some embodiments, the programmed sequence comprises a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

In some embodiments, the feedback mechanism is performed by either hardware or software. Preferably, the feedback mechanism is performed by an analog closed-loop circuit. Preferably, this feedback occurs every 50 μ8, 20 μ8, 10 μ8 or 1 μ8, but is preferably a real-time feedback or instantaneous (i.e., substantially instantaneous as determined by available techniques for determining response time). In some embodiments, the neutral electrode measures the impedance in the desired tissue and communicates the impedance to the feedback mechanism, and the feedback mechanism responds to the impedance and adjusts the pulse of energy to maintain the constant current at a value similar to the preset current. In some embodiments, the feedback mechanism maintains the constant current continuously and instantaneously during the delivery of the pulse of energy.

In some embodiments, the nucleic acid molecule is delivered to the cells in conjunction with administration of a polynucleotide function enhancer or a genetic vaccine facilitator agent. Polynucleotide function enhancers are described in U.S. Pat. Nos. 5,593,972, 5,962,428 and International Application Serial Number PCT/US94/00899 filed Jan. 26, 1994, which are each incorporated herein by reference. The co-agents that are administered in conjunction with nucleic acid molecules may be administered as a mixture with the nucleic acid molecule or administered separately simultaneously, before or after administration of nucleic acid molecules. In addition, other agents which may function transfecting agents and/or replicating agents and/or inflammatory agents and which may be co-administered with a GVF include growth factors, cytokines and lymphokines such as a-interferon, gamma-interferon, GM-CSF, platelet derived growth factor (PDGF), TNF, epidermal growth factor (EGF), IL-1, IL-2, IL-4, IL-6, IL-10, IL-12 and IL-15 as well as fibroblast growth factor, surface active agents such as immune-stimulating complexes (ISCOMS), Freunds incomplete adjuvant, LPS analog including monophosphoryl Lipid A (WL), muramyl peptides, quinone analogs and vesicles such as squalene and squalene, and hyaluronic acid may also be used administered in conjunction with the genetic construct In some embodiments, an immunomodulating protein may be used as a GVF. In some embodiments, the nucleic acid molecule is provided in association with PLG to enhance delivery/uptake.

The pharmaceutical compositions according to the present invention comprise about 1 nanogram to about 2000 micrograms of DNA. In some preferred embodiments, pharmaceutical compositions according to the present invention comprise about 5 nanogram to about 1000 micrograms of DNA. In some preferred embodiments, the pharmaceutical compositions contain about 10 nanograms to about 800 micrograms of DNA. In some preferred embodiments, the pharmaceutical compositions contain about 0.1 to about 500 micrograms of DNA. In some preferred embodiments, the pharmaceutical compositions contain about 1 to about 350 micrograms of DNA. In some preferred embodiments, the pharmaceutical compositions contain about 25 to about 250 micrograms of DNA. In some preferred embodiments, the pharmaceutical compositions contain about 100 to about 200 microgram DNA. In certain embodiments, the VGX-3100 is formulated at a concentration of 6 mg/ml.

The pharmaceutical compositions according to the present invention are formulated according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation. In some preferred embodiments, the pharmaceutical compositions contain about 100 to about 200 microgram DNA. In certain embodiments, the VGX-3100 is formulated at a concentration of 6 mg/ml in 150 mM sodium chloride and 15 mM sodium citrate.

In certain embodiments, the VGX-3100 is administered to the subject at a dose of 6 mg. In further embodiments, the VGX-3100 is administered to the subject three times over the course of 12 weeks. In still further embodiments, a first dose of VGX-3100 is administered on Day 0, the second dose of VGX-3100 is administered at Week 4, and the third dose of VGX-3100 is administered at Week 12.

### EXAMPLES

The example and embodiments described herein are for illustrative purposes only and various modifications or changes suggested to persons skilled in the art are to be included within the spirit and purview of this application and scope of the appended claims.

### Example 1: Reveal 2—A Randomized, Double-Blind, Placebo-Controlled Phase 3 Study of VGX-3100 Delivered Intramuscularly Followed by Electroporation with Cellectra™ 5PSP for the Treatment of HPV-16 and/or HPV-18 Related High Grade Squamous Intraepithelial Lesion (HSIL) of the Cervix (HPV-303)

A non-limiting example of a randomized, double-blind, placebo-controlled phase 3 Study of VGX-3100 delivered intramuscularly (IM) followed by electroporation with CELLECTRA™ 5PSP for the Treatment of HPV-16 and/or HPV-18 related high grade squamous intraepithelial lesion (HSIL) of the cervix is provided herein. The primary endpoint was the proportion of baseline biomarker-positive women with no evidence of cervical HSIL on histology sample and no evidence of HPV-16 and/or HPV-18 in cervical samples by type specific HPV testing at Week 36 visit.

### Clinical Trial Objectives and Endpoints:

Primary Objective: Among baseline biomarker-positive women, determine the efficacy of VGX-3100 compared with placebo with respect to combined histopathologic regression of cervical HSIL and virologic clearance of HPV-16 and/or HPV-18.

Primary Endpoint: Proportion of baseline biomarker-positive women with no evidence of cervical HSIL on histology sample and no evidence of HPV-16 and/or HPV-18 in cervical samples by type specific HPV testing at Week 36 visit.

The secondary objectives and associated secondary endpoints are provided in Table 1.

TABLE 1

| Secondary Objectives | | Associated Secondary Endpoints | |
|---|---|---|---|
| 1. | Among i) baseline biomarker-positive women | 1a. | Incidence and severity of local and systemic |
| | and ii) all women, evaluate the safety and | | events for 7 and 28 days following each |
| | tolerability of VGX-3100 delivered IM followed | | investigational treatment and for the duration |
| | by EP with CELLECTRA ™ 5PSP. | | of the study (i.e., 40 weeks). |
| | | 1b. | Incidence and severity of all adverse events |
| | | | including Serious adverse events (SAEs) (e.g., |
| | | | Serious unexpected serious adverse reaction |
| | | | (SUSAR), Unexpected adverse device effect |
| | | | (UADE) and other unexpected AEs) for the |
| | | | duration of the study (through Week 40 visit). |
| 2. | Among all women, determine the efficacy of | 2. | Proportion of all women with no evidence of |
| | VGX-3100 compared with placebo with respect | | cervical HSIL on histology (i.e., biopsies or |
| | to combined histopathologic regression of | | excisional treatment) and no evidence of HPV- |
| | cervical HSIL and virologic clearance of HPV-16 | | 16 and/or HPV-18 in cervical samples by type |
| | and/or HPV-18. | | specific HPV testing at Week 36 visit. |
| 3. | Among i) baseline biomarker-positive women | 3. | Proportion of women with no evidence of |
| | and ii) all women, determine VGX-3100 efficacy | | cervical HSIL on histology (i.e., biopsies or |
| | compared to placebo as measured by | | excisional treatment) at Week 36 visit. |
| | histopathologic regression of cervical HSIL. | | |
| 4. | Among i) baseline biomarker-positive women | 4. | Proportion of women with no evidence of |
| | and ii) all women, determine VGX-3100 efficacy | | HPV-16 and/or HPV-18 in cervical samples by |
| | compared to placebo as measured by virologic | | type specific HPV testing at Week 36 visit. |
| | clearance of HPV-16 and/or HPV-18. | | |
| 5. | Among i) baseline biomarker-positive women | 5. | Proportion of women with no evidence of Low |
| | and ii) all women, determine VGX-3100 efficacy | | grade squamous intraepithelial lesion (LSIL) or |
| | compared to placebo as measured by complete | | HSIL (i.e., no evidence of CIN1, CIN2 or |
| | histopathologic regression of cervical HSIL to | | CIN3) on histology (i.e., biopsies or excisional |
| | normal. | | treatment) at Week 36 visit. |
| 6. | Among i) baseline biomarker-positive women | 6. | Proportion of women with no evidence of LSIL |
| | and ii) all women, determine VGX-3100 efficacy | | or HSIL (i.e., no evidence of CIN1, CIN2 or |
| | compared to placebo as measured by both | | CIN3 on biopsies or excisional treatment) on |
| | complete histopathologic regression of cervical | | histology (i.e., biopsies or excisional treatment) |
| | HSIL to normal and virologic clearance of HPV- | | and no evidence of HPV-16 and/or HPV-18 by |
| | 16 and/or HPV-18. | | type specific HPV testing at Week 36 visit. |
| 7. | Among i) baseline biomarker-positive women | 7. | Proportion of women with no progression of |
| | and ii) all women, determine VGX-3100 efficacy | | cervical HSIL to cervical carcinoma from |
| | compared to placebo as measured by | | baseline on histology (i.e., biopsies or |
| | histopathologic non-progression. | | excisional treatment) at Week 36 visit. |
| 8. | Among i) baseline biomarker-positive women | 8. | Proportion of women who have cleared HPV- |
| | and ii) all women, describe the clearance of | | 16 and/or HPV-18 on specimens from non- |
| | HPV-16 and/or HPV-18 infection from non- | | cervical anatomic locations (oropharynx, |
| | cervical anatomic locations. | | vagina and intra-anal) at Week 36 Visit |
| | | | compared to baseline. |
| 9. | Among i) baseline biomarker-positive women | 9a. | Levels of serum anti-HPV-16 and anti-HPV-18 |
| | and ii) all women, determine the humoral and | | antibody concentrations at Weeks 15 and 36 |
| | cellular immune response of VGX-3100 | | visits. |
| | compared with placebo at post dose 3 and week | 9b. | Interferon-y ELISpot response magnitudes at |
| | 36 visit as assessed relative to baseline. | | baseline, Weeks 15 and 36 visits. |
| | | 9c. | Flow Cytometry response magnitudes at |
| | | | baseline and Week 15 visits. |

The exploratory objectives and associated exploratory endpoints are provided in Table 2.

TABLE 2

| Exploratory Objectives | | Associated Exploratory Endpoints | |
|---|---|---|---|
| 1. | Among i) baseline biomarker-positive women | 1. | Assessment of markers including but not |
| | and ii) all women, evaluate tissue immune | | limited to CD8+ and FoxP3+ infiltrating cells. |
| | responses to VGX-3100 in cervical samples. | | Additional assessments may include |
| | | | visualization of Granulysin, Perforin, CD137, |
| | | | CD103 and PD-L1 in cervical tissue as sample |
| | | | allows. Markers listed here may change as |
| | | | new relevant information becomes available. |
| 2. | Describe association of microRNA (miRNA) | 2. | Colposcopy, cytology, and HPV test results |
| | profiles, DNA methylation profile, previous | | (Weeks 8, 15 and 28 visits), miRNA profile |
| | colposcopy, cytology and HPV testing results | | (baseline, Week 8) and DNA methylation |
| | with Week 36 histologic regression. | | profile (baseline, Week 15) in conjunction with |
| | | | histologic regression of cervical HSIL at Week |
| | | | 36 visit. |
| 3. | Describe the association of a tissue-based score | 3. | Immunoscore results for VGX-3100 in |
| | derived using immunologic markers | | conjunction with histological and virological |
| | (Immunoscore) at baseline to histological and | | outcomes at Week 36. |
| | virological response to VGX-3100 at Week 36. | | |

### Study Design

Trial Treatment: 6 mg (1 ml) VGX-3100 Intramuscular injection followed by EP with the CELLECTRAT 5PSP device given at Day 0, Week 4 and Week 12.

Study Design: To be eligible for the study, subjects at least 18 years age and above the age of consent must have agreed to participate and have cervical biopsy/biopsies of the cervical lesion(s) at the time of Screening. Slides of the biopsy were sent to the PAC in a blinded manner to establish the presence of cervical HSIL within screening. In order to be eligible for randomization, the PAC must assign the histologic diagnosis of cervical HSIL. Subjects must also have a cervical specimen test positive for HPV-16/18 by Cobas™ HPV test to be eligible for participation in the study.

A sample of approximately 198 subjects were randomized to receive either 6 mg (in 1 ml) VGX-3100 or placebo, each IM followed by EP, in a 2:1 ratio. This sample size provides >90% power to declare VGX-3100 superior to placebo among biomarker-positive women, assuming, based upon HPV-301 study results, that the true proportion of subjects whose lesion(s) regress and whose HPV-16/18 clear is 66% and 15% for VGX-3100 and placebo, respectively, and that the proportion of biomarker-positive women is 33%. Biomarker positivity in this context is defined as being predicted to exhibit lesion regression concomitant with HPV16/18 clearance after interrogation of a pre-defined microRNA (miRNA) signature in subject plasma which is assessed prior to the administration of VGX-3100 or placebo. Those subjects who are not predicted to exhibit lesion regression and HVP16/18 clearance based on this signature prior to dosing with VGX-3100 or placebo are considered biomarker-negative.

Subjects were randomized in a stratified manner according to (a) the CIN severity observed in the biopsy specimens at screening (i.e. CIN2 vs. CIN3), (b) BMI category (≤25 vs. >25 kg/m2) on Day 0, and (c) age category (<25 years vs. ≥25 years) on Day 0. To ensure CIN2 disease is not over-represented in the study, the percentage of subjects enrolled with CIN2 did not exceed 50% of the total enrolled. Each country received a group of sequential allocation numbers.

Efficacy Assessment: The primary endpoint for the Phase 3 study (HPV-303) is based upon the results of the Phase 2b study (HPV-003) and the Phase 3 study (HPV-301). Given that HPV persistence is an important factor in the clinical progression of cervical dysplasia and also based upon the findings of the secondary objective of the HPV-003 study, the responder definition for the HPV-303 primary endpoint determination will take into consideration both histological regression of cervical HSIL and clearance of high-risk HPV-16 and HPV-18.

Regression of cervical HSIL was determined by histopathological assessment of cervical tissue, which is considered the definitive method for diagnosis of cervical dysplasia. Tissue to be analyzed for evidence of histopathologic regression will be obtained at Week 36. Cervical cytology samples were obtained to characterize HPV infection at screening. Day ((prior to dosing) and Weeks 8, 15, 28 and 36. Also, if there is residual tissue in the paraffin block after histologic diagnoses have been rendered, then unstained slides and/or the relevant paraffin blocks may be collected for testing of HPV-16 and/or HPV-18. Vaginal, oropharyngeal, and optional intra-anal samples will be obtained to characterize HPV infection at Day 0 (prior to dosing) and at Week 36 to assess virologic response to treatment at sites other than the cervix.

Immunogenicity Assessment: Humoral and cell mediated immune responses in response to VGX-3100 treatment were evaluated in blood samples taken at baseline (both Screening as well as Day 0 prior to dosing) and at Weeks 15, and 36. Cervical tissue samples were analyzed for evidence of elevated immune responses at Week 36 as compared to baseline (Screening).

Safety Assessment: The risk of HSIL progression to cancer or recurrence of HSIL is considered low and comparable to the rates observed post-LEEP/CKC. The long-term follow-up planned for this HPV-303 study will include safety, cytology and HPV-16 and/or HPV-18 testing up to 7 months following the last dose of Study Treatment.

A Data Safety Monitoring Board (DSMB) will review safety data and histopathological regression results. The DSMB will be charged with advising the Sponsor if there appears to be a safety issue and if it appears that the proportion of the subjects with histopathologic regression in the VGX-3100 group is unacceptably low compared to the placebo group. No formal interim analysis will be performed.

Trial Population: Women 18 years of age and older were recruited in this multi-center global trial. VGX-3100 is being developed as a non-surgical therapeutic option for the treatment of the precursor to cervical cancer, precancerous cervical HSIL, and the underlying pathogenic HPV-16 and/or HPV-18 infection.

Inclusion Criteria—Each subject must meet all of the following criteria to be enrolled in the study:
1. Women aged 18 years and above that meet the minimum age of consent per local regulations:
2. Confirmed cervical infection with HPV types 16 and/or 18 at screening by Cobas™ HPV test:
3. Cervical tissue specimen/slides provided to Study Pathology Adjudication Committee for diagnosis must be collected within 10 weeks prior to anticipated date of first dose of study drug:
4. Histologic evidence of cervical HSIL as confirmed by Pathological Adjudication Committee (PAC) at screening:
5. Must understand, agree and be able to comply with the requirements of the protocol; Subjects must be willing and able to provide voluntary consent to participate and sign a Consent Form prior to study related activities:
6. Must be judged by investigator to be an appropriate candidate for the protocol-specified procedure(s) required at Week 36;
7. Satisfactory colposcopy at screening, defined as full visualization of the squamo-columnar junction (Type I or II transformation zone) and complete visualization of the upper limit of aceto-white epithelium or suspected CIN disease;
8. Cervical lesion that is accessible for sampling by biopsy instrument (e.g. Mini-Tischler device):
9. Cervical lesion of adequate size to ensure that a visible lesion remains after screening biopsy:
10. Must meet one of the following criteria with respect to their reproductive capacity:
   a. Post-menopausal as defined by spontaneous amenorrhea for more than 12 months
   b. Surgically sterile due to absence of ovaries or due to a bilateral tubal ligation/occlusion performed more than 12 months prior to screening:
   c. Women of Child Bearing Potential (WOCBP) is willing to use a contraceptive method with failure rate of less than 1% per year when used consistently and correctly from screening until Week 36. Use of condoms alone or condoms with spermicide does not have a failure rate of <1% per year and is therefore not an acceptable form of contraception The following methods are acceptable:
      Hormonal contraception: either combined or progestin-alone including oral contraceptives, injectable, implants, vaginal ring, or percutaneous patches. Hormonal contraceptives must not be used in subjects with a history of hypercoagulability (e.g., deep vein thrombosis, pulmonary embolism):
      Abstinence from penile-vaginal intercourse when this is the subject's mode of sexual activity:
      Intrauterine device or intrauterine system:
      Male partner sterilization at least 6 months prior to the female subject's entry into the study, and this male is the sole partner for that subject.
11. Normal screening Electrocardiogram (ECG) or screening ECG with no clinically significant findings, as judged by the investigator.

Exclusion Criteria-Subjects meeting any of the following criteria will be excluded from enrollment or continuation in the study:
1. Microscopic or gross evidence of adenocarcinoma-in-situ (AIS), high grade vulvar, vaginal (inclusive of cervical HPV-related lesions that extend into the vaginal vault), or anal intraepithelial neoplasia or invasive cancer in any histopathologic specimen at screening:
2. Cervical lesion(s) that cannot be fully visualized on colposcopy due to extension high into cervical canal at screening:
3. ECC that shows a potentially untreated carcinoma, untreated HSIL, indeterminate, or insufficient for diagnosis (ECC is not required to be performed as part of study screening);
4. Treatment for cervical HSIL within 4 weeks prior to screening:
5. Pregnant, breastfeeding or considering becoming pregnant through week 36 visit:
6. History of previous therapeutic HPV vaccination (licensed prophylactic HPV vaccines are allowed, e.g. Gardasil™, Silgard™. Cervarix™);
7. Presence of any unresolved abnormal clinical screening laboratory values of Grade 1 or greater per Common Toxicity Criteria for Adverse Events (CTCAE) v 4.03 and deemed clinically significant by the investigator 60 days prior to Day 0:
8. Immunosuppression as a result of underlying illness or treatment including:
   a) History of or positive serologic test for HIV at screening (performed within 60 days prior to Day 0):
   b) Primary immunodeficiencies:
   c) Long term use (≥7 days) of oral or parenteral glucocorticoids at a dose of ≥20 mg/day of prednisone equivalent: (use of inhaled, otic, and ophthalmic corticosteroids are allowed):
   d) Current or anticipated use of disease modifying doses of anti-rheumatic drugs (e.g., azathioprine, cyclophosphamide, cyclosporine, methotrexate), and biologic disease modifying drugs such as TNF-α inhibitors (e.g. infliximab, adalimumab or etanercept);
   e) History of solid organ or bone marrow transplantation:
   f) Any prior history of other clinically significant immunosuppressive or clinically diagnosed autoimmune disease that may jeopardize the safety of the subject or require therapy that would interfere with study assessments or endpoint evaluation, or otherwise impact the validity of the study results:
   g) Subjects who are malnourished (i.e. medically significant unintentional weight loss) based on screening labs, medical history and physical exam per the investigator's clinical judgment.
9. Receipt of any non-study, non-live vaccine within 2 weeks of Dosing:
10. Receipt of any non-study, live vaccine (e.g. measles vaccine) within 4 weeks of Dosing:
11. Current or history of clinically significant, medically unstable disease which, in the judgment of the investigator, would jeopardize the safety of the subject, interfere with study assessments or endpoint evaluation, or otherwise impact the validity of the study results (e.g. chronic renal failure; angina, myocardial ischemia or infarction, class 3 or higher congestive heart failure, cardiomyopathy, or clinically significant arrhythmias);
12. Malignancy or systemic treatment for malignancy within 2 years of screening (locally treated ano-genital malignancy and superficial skin cancers are allowed);
13. Presence of acute or chronic bleeding or clotting disorder that would contraindicate IM injections, or use of blood thinners (e.g. anticoagulants or antiplatelet drugs) within 2 weeks of Day 0;
14. History of seizures unless seizure free for 5 years with the use of one or fewer antiepileptic agents:
15. Sustained, manually confirmed, sitting systolic blood pressure >150 mm Hg or <90 mm Hg or a diastolic blood pressure >95 mm Hg at Screening or Day 0:
16. Resting heart rate <50 bpm (unless attributable to athletic conditioning) or >100 bpm at screening or Day 0:
17. Prior major surgery within 4 weeks of Day 0;
18. Participation in an interventional study with an investigational compound or device (except HPV-301 REVEAL 1) within 30 days of signing informed consent: participation in an observational study is permitted:
19. Less than two acceptable sites available for IM injection considering the deltoid and anterolateral quadriceps muscles:
20. Tattoos, keloids or hypertrophic scars located within 2 cm of intended treatment site:
21. Cardioverter-defibrillator or pacemaker (to prevent a life-threatening arrhythmia) that is located in ipsilateral deltoid injection site (unless deemed acceptable by a cardiologist);
22. Metal implants or implantable medical device within the electroporation area:
23. Active drug or alcohol use or dependence that, in the opinion of the investigator, would interfere with adherence to study requirements:
24. Prisoner or subject who is compulsorily detained (involuntarily incarcerated) for treatment of either a psychiatric or physical (i.e. infectious disease) illness:
25. Active military service personnel:
26. Study-related staff or family member of study-related staff:
27. Any illness or condition that in the opinion of the investigator may affect the safety of the subject or the evaluation of any study endpoint.
   The definition of composite regression and clearance endpoint responder and non-responder and provided in Table 3:

TABLE 3

| Responder | Non-Responder |
|---|---|
| Subject with no histologic evidence of cervical | Subject with histologic evidence of cervical HSIL, AIS, |
| HSIL^{a} at Week 36 evaluation and no evidence | cervical carcinoma at Week 36 visit |
| of HPV-16 and/or HPV-18 at Week 36 visit | OR |
| AND | Subject with evidence of HPV-16 or HPV-18 at Week 36 |
| Subject in whom an excision or biopsy sample^{c} | visit |
| was NOT obtained between initial dose up to | OR |
| Week 36 visit | Subject in whom an excision or biopsy sample was |
| | obtained between initial dose up to Week 36 visit |
| | OR |
| | Subjects with no Week 36 visit sample result |

### Test Product, Dose and Mode of Administration, Batch Number(s)

### Investigational Product

Common name: VGX-3100

Chemical name: Circular, double stranded, deoxyribonucleic acid consisting of 3782 base pairs for the pGX3001 plasmid and 3824 base pairs for the pGX3002 plasmid.

Distinguishing name: Eukaryotic expression plasmids containing HPV 16 and 18-E6 & E7-encoding transcription unit controlled by a synthetic, CMV promoter, and elements required for replication and selection in E. coli, namely a pUC origin of replication (pUC Ori) and a kanamycin resistance gene (Kan R).

Detailed description: VGX-3100, HPV therapeutic vaccine is a combination of two plasmids in equal quantities (i.e. the 6 mg dose will deliver 3 mg of each pGX3001 and pGX3002 plasmids): a) pGX3001: p16ConE6E7, a plasmid encoding for a synthetic HPV16 consensus E6 and E7 fusion gene (“consensus HPV 16-6&7”) into a pVAX1 backbone (Invitrogen, Carlsbad, CA) under the control of the cytomegalovirus immediate-early (CMV) promoter, and b) pGX3002: p18ConE6E7, a plasmid encoding for a synthetic HPV18 consensus E6 and E7 fusion gene (“consensus HPV 18-6&7”) into a pVAX1 backbone (Invitrogen, Carlsbad, CA) under the control of the cytomegalovirus immediate-early (CMV) promoter. VGX-3100 is described in WO2014/165291, which is incorporated herein by reference in its entirety. The nucleic acid and amino acid consensus sequences are provided in Table 4 and Table 5, respectively.

TABLE 4
VGX-3100 consensus sequences (nucleic acid)

| Consensus | | |
|---|---|---|
| Fusion | | SEQ ID |
| Gene | Nucleic Acid Sequence | NO |
| Consensus | gaattcgccaccatggactggacctggatcctgttcctggtggccgccgccacacgggtgcacagcttcc | 1 |
| HPV 16- | aggacccccaggagagcggcagaaagctgcctcagctgtgtaccgagctgcagaccaccatccacgac | |
| 6 & 7 | atcatcctggagtgtgtgtactgtaagcagcagctgctgaggagagaggtgtacgaccgggacctgtgtat | |
| | cgtgtacagggacggcaatccctacgccgtgtgtgacaagtgcctgaagttctacagcaagatcagcgag | |
| | taccggcactactgctacagcctgtacggcaccaccctggagcagcagtacaacaagcccctgtgtgac | |
| | ctgctgatccggtgtatcaactgccagaagcccctgcagagacacctggacaagaagcagcggttccac | |
| | aacatcaggggcagatggaccggcagatgtatgagctgctgccggagcagcagaaccagaagggaga | |
| | cccagctgagaggccggaagagaagaagccacggcgatacccccaccctgcacgagtacatgctgga | |
| | cctgcagcctgagaccaccgatctgtacggctacggccagctgaatgacagcagcgaggaggaggatg | |
| | agatcgacggccctgccggccaggccgagcccgacagagcccactacaacatcgtgaccttttgctgta | |
| | agtgtgacagcaccctgagactgtgcgtgcagagcacccacgtggacatcagaaccctggaggatctgc | |
| | tgatgggcaccctgggcatcgtgtgtcccatctgctcccagaaaccctgatgagcggccgc | |
| Consensus | atggactggacctggatcctgttcctggtggccgctgccacacgggtgcacagcgccagattcg | 2 |
| HPV 18- | aggaccccaccaggagcggctacaagctgcccgatctgtgtaccgagctgaacaccagcctgc | |
| 6 & 7 | aggacatcgagatcacctgtgtgtactgtaagaccgtgctggagctgaccgaggtgttcgagaa | |
| | ggacctgttcgtggtgtacagggacagcatcccccacgccgcctgccacaagtgtatcgacttct | |
| | acagccggatccgggagctgagacactacagcgacagcgtgtacggcgataccctggagaag | |
| | ctgaccaacaccggcctgtacaacctgctgatccggtgcctgagatgccagaagcccctgctga | |
| | gacacctgaacgagaagcggcggttccacaacatcgccggccactacagaggccagtgccac | |
| | agctgctgtaacagggccaggcaggagagactgcagcggagaagagagacccaggtgagg | |
| | ggcaggaagagaagaagccacggccccaaggccaccctgcaggatatcgtgctgcacctgg | |
| | agccccagaatgagatccccgtggatctgctgggccacggccagctgtccgacagcgaggag | |
| | gagaacgacgagatcgacggcgtgaatcaccagcacctgcctgccagaagagccgagcctca | |
| | gaggcacaccatgctgtgtatgtgctgtaagtgtgaggcccggatcgaactggtggtggagagc | |
| | agcgccgacgacctgagagccttccagcagctgttcctgaacaccctgagcttcgtgtgtccttg | |
| | gtgtgccagccagcagtga | |

TABLE 5
VGX-3100 consensus sequences (amino acid)

| Consensus | | |
|---|---|---|
| Fusion | | SEQ ID |
| Gene | Amino Acid Sequence | NO |
| Consensus | MDWTWILFLVAAATRVHSFQDPQESGRKLPQLCTELQTTIHDIILE | 3 |
| HPV 16- | CVYCKQQLLRREVYDRDLCIVYRDGNPYAVCDKCLKFYSKISEYR | |
| 6 & 7 | HYCYSLYGTTLEQQYNKPLCDLLIRCINCQKPLQRHLDKKQRFHNI | |
| | RGRWTGRCMSCCRSSRTRRETQLRGRKRRSHGDTPTLHEYMLDL | |
| | QPETTDLYGYGQLNDSSEEEDEIDGPAGQAEPDRAHYNIVTFCCKC | |
| | DSTLRLCVQSTHVDIRTLEDLLMGTLGIVCPICSQKP | |
| Consensus | MDWTWILFLVAAATRVHSARFEDPTRSGYKLPDLCTELNTSLQDI | 4 |
| HPV 18- | EITCVYCKTVLELTEVFEKDLFVVYRDSIPHAACHKCIDFYSRIREL | |
| 6 & 7 | RHYSDSVYGDTLEKLTNTGLYNLLIRCLRCQKPLLRHLNEKRRFH | |
| | NIAGHYRGQCHSCCNRARQERLQRRRETQVRGRKRRSHGPKATL | |
| | QDIVLHLEPQNEIPVDLLGHGQLSDSEEENDEIDGVNHQHLPARRA | |
| | EPQRHTMLCMCCKCEARIELVVESSADDLRAFQQLFLNTLSFVCP | |
| | WCASQQ | |

In preparation for the Phase 3 program, a buffered refrigerated (2-8° C. storage) formulation of VGX 3100 was developed using a saline sodium citrate (SSC) solution. The refrigerated formulation requires administration using the next generation device, the CELLECTRA™ 5PSP. This refrigerated formulation of VGX-3100 was first administered to 117 subjects in a Phase 1 clinical trial, HPV-101. Roughly half the healthy volunteers in the HPV-101 study, received three 6 mg IM doses of VGX-3100 (refrigerated formulation) followed by EP. Based upon an analysis of the data, the refrigerated formulation was determined to be non-inferior to the frozen formulation based upon a 2-fold rise in overall Spot Forming Units (SPU) to the antigens encoded by the plasmids per 106 peripheral blood mononuclear cells (PBMC) as measured from baseline to Week 14 using the interferon-γ ELISpot assay.

### Efficacy Results

The trial did not meet the primary endpoint of regression and clearance in biomarker-positive population. The trial did meet the secondary endpoint of regression and clearance in all patients. VGX-3100 was tolerable and there was no difference in the reported AE profile between treatment and placebo arms. VGX-3100 was immunogenic and created antigen-specific T cells in the periphery and created T cells in the tissue.

The study population is provided in Table 6:

TABLE 6

| | VGX-3100 | Placebo | Total | |
|---|---|---|---|---|
| | (N) | (N) | (N) | |
| All Subjects | 134 | 69 | 203 | (100%) |
| Biomarker-positive subjects | 21 | 4 | 25 | (12.3%) |

Regression of HSIL and clearance of virus is provided in Table 7:

TABLE 7

| Regression of HSIL | | | | Difference in Effect |
|---|---|---|---|---|
| and Clearance | | | | Seen (95% Confidence |
| of Virus | VGX-3100 | | Placebo | Interval; p-value) |
| Biomarker | 6 | (28.6%) | 0 | 28.6 (−24.6, 50.4; |
| (Primary) | | | | 0.115) |
| All Patients | 37 | (27.6%) | 6 (8.7%) | 18.9 (7.8, 28.6; |
| (Secondary) | | | | 0.001) |

Regression of HSIL or clearance of virus is provided in Table 8

TABLE 8

| | VGX-3100 | | Placebo | | Difference (95% CI) | |
|---|---|---|---|---|---|---|
| Regression | | | | | | |
| Biomarker | 8 | (38.1) | 1 | (25) | 13.1 | (−37.7, 46) |
| All Patients | 46 | (34.3) | 15 | (21.7) | 12.6 | (−0.8, 24.5) |
| Clearance | | | | | | |
| Biomarker | 7 | (33.3) | 0 | | 33.3 | (−20.1, 55) |
| All Patients | 50 | (37.3) | 6 | (8.7) | 28.6 | (17.1, 38.7) |

The adverse events reported in >10% of patients showed a favorable safety profile with no difference between VGX-3100 and placebo arms. Adverse events are reported in Table 9.

TABLE 9

| Adverse Event | VGX-3100 (%) | Placebo (%) |
|---|---|---|
| Injection site pain | 91.8 | 86.6 |
| Headache | 49.3 | 55.2 |
| Fatigue | 41.8 | 49.3 |
| Myalgia | 41.8 | 43.3 |
| Injection site swelling | 32.1 | 28.4 |
| Nausea | 26.9 | 25.4 |
| Injection site pruritis | 24.6 | 32.8 |
| Arthralgia | 21.6 | 20.9 |
| Injection site bruising | 15.7 | 16.4 |
| Malaise | 13.4 | 17.9 |
| Injection site hematoma | 7.5 | 10.4 |

Reported events of progression to cancer is provided in Table 10.

TABLE 10

| | | |
|---|---|---|
| Progression from | VGX-3100 | Placebo |
| HSIL to Cancer | (N, %) | (N, %) |

| | | | |
|---|---|---|---|
| Biomarker | 3 | (14%) | 0 |
| All Patients | 8 | (6%) | 7 (10%) |

Conclusions: While not achieving the primary endpoint of regression and clearance in the biomarker-positive population, the trial met the endpoint of regression and clearance in the total population (secondary). The total number of progression cases was 15 (7%), with 8 (6%) on treatment arm and 7 (10%) on placebo arm. Adverse event profile was statistically equivalent across arms. There was evidence of T cell generation.

What is claimed:

1. A method of treating human papillomavirus (HPV) type 16- or HPV type 18-related high grade cervical intraepithelial lesion (HSIL), said method comprising administering a therapeutically effective amount of VGX-3100 to a subject in need thereof.

2. The method of claim 1, wherein HPV type 16- or HPV type 18-related HSIL is determined by a biopsy.

3. The method of claim 1, wherein VGX-3100 is administered to the subject by intramuscular injection followed by electroporation.

4. The method of claim 1, wherein VGX-3100 is administered to the subject at a dose of 6 mg.

5. The method of claim 1, wherein VGX-3100 is administered to the subject three times over the course of 12 weeks.

6. The method of claim 1, wherein VGX-3100 is formulated at a concentration of 6 mg/ml in 150 mM sodium chloride and 15 mM sodium citrate.

7. The method of claim 1, wherein administration of VGX-3100 results in virologic clearance of HPV-16 and/or HPV-18 and histopathologic regression of cervical HSIL.

8. The method of claim 1, wherein administration of VGX-3100 results in histopathologic regression of cervical HSIL.

9. The method of claim 1, wherein administration of VGX-3100 results in virologic clearance of HPV-16 and/or HPV-18.

10. The method of claim 1, wherein administration of VGX-3100 results in complete histopathologic regression of cervical HSIL to normal.

11. The method of claim 1, wherein administration of VGX-3100 results in complete histopathologic regression of cervical HSIL to normal and virologic clearance of HPV-16 and/or HPV-18.

12. The method of claim 1, wherein administration of VGX-3100 results in histopathologic non-progression.

13. The method of claim 1, wherein administration of VGX-3100 results in clearance of HPV-16 and/or HPV-18 infection from noncervical anatomic locations.

14. The method of claim 1, wherein administration of VGX-3100 results in improved humoral and cellular immune response to VGX-3100 following a third administration of VGX-3100 and at 36 weeks following administration of VGX-3100 as assessed relative to baseline.

15. The method of claim 1, wherein the result of VGX-3100 administration is evaluated at 36 weeks following administration of VGX-3100.
